(19) United States
(12) Patent Application Publication
(10) Pub. No.: US 2025/0331776 A1
(43) Pub. Date: Oct. 30, 2025

(54) IMPLANTABLE DATA LOGGER ANCHORING DEVICES

(71) Applicant: The Jackson Laboratory, Bar Harbor, ME (US)

(72) Inventors: Kevin Kane, Bar Harbor, ME (US); Jarek Trapszo, Bar Harbor, ME (US); Rebecca LaFleur, Bar Harbor, ME (US); Andree LaPierre, Bar Harbor, ME (US)

(73) Assignee: The Jackson Laboratory, Bar Harbor, ME (US)

(21) Appl. No.: 18/865,074

(22) PCT Filed: May 12, 2023
(86) PCT No.: PCT/US2023/021979

§ 371 Date: Nov. 12, 2024

Related U.S. Application Data
(60) Provisional application No. 63/341,722, filed on May 13, 2022.

(51) Int. Cl. A61B 5/00

(52) U.S. Cl. CPC A61B 5/6882; A61B 5/686; A61B 2562/162

(57) ABSTRACT

An anchoring device may include a body including a cavity configured to receive a data logger disposed therein. An anchoring device may include an anchoring portion attached to the body. An anchoring device may include one or more through holes formed in the anchoring portion, where the one or more through holes are configured to accept a suture passing therethrough to attach the anchoring device to tissue of a subject.

### RELATED APPLICATIONS

This application claims the benefit of priority under 35 U.S.C. § 119 (e) to U.S. Provisional Application No. 63/341,722, filed May 13, 2022, which is herein incorporated by reference in its entirety.

### FIELD

Disclosed embodiments are related to anchoring devices for implantable data loggers and related methods of use.

### BACKGROUND

Data loggers are electronic devices that record data over time either with a built-in instrument or sensor or via external instruments and sensors. Some devices may be implanted in the abdomen of animal models, for example, rodent models.

### SUMMARY

Some aspects provide anchoring devices designed to fit an implantable data logger. Implantable data loggers are often designed to move freely in the abdominal cavity, but there are downsides to having the implantable data logger freely movable. Thus, provided herein, in some aspects, are anchoring devices designed to immobilize (anchor) the implantable data logger within the abdomen of a laboratory animal, for example, a rodent such as a mouse.

Some aspects provide an anchoring device comprising: a body including a cavity configured to receive a data logger disposed therein, an anchoring portion attached to the body, and one or more through holes formed in the anchoring portion, where the one or more through holes are configured to accept a suture passing therethrough to attach the anchoring device to tissue of a subject.

In some embodiments, the cavity is a cylindrical cavity extending through the body, and wherein the cavity is configured to form a friction fit with the data logger.

In some embodiments, the body is sufficiently elastic such that the body may be deformed to position the data logger in the cavity.

In some embodiments, the anchoring device further comprises the data logger disposed in the cavity.

In some embodiments, the anchoring portion extends outwards from the body.

In some aspects, the embodiments described herein relate to a data logger anchoring device including a body including a first end and a second end configured to join together when disposed around a data logger, an anchoring portion attached to the body, and one or more through holes formed in the anchoring portion, where the one or more through holes are configured to accept a suture passing therethrough to attach the data logger anchoring device to tissue of a subject.

### BRIEF DESCRIPTION OF THE DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A shows a drawing of an embodiment of an anchoring device applied to a data logger device;

FIG. 1B shows a drawing of the anchoring device of FIG. 1A without a data logger device;

FIG. 2A shows a drawing of an embodiment of an anchoring device applied to a data logger device;

FIG. 2B shows a drawing of the anchoring device of FIG. 2A without a data logger device;

FIG. 3A shows a top view of an embodiment of an anchoring device;

FIG. 3B shows a side view of the anchoring device of FIG. 3A;

FIG. 4A shows a side view of an embodiment of an anchoring device;

FIG. 4B shows an opposing side view of the anchoring device of FIG. 4A;

FIG. 5A shows a perspective view of an embodiment of an anchoring device applied to a data logger device; and

FIG. 5B shows a second perspective view of the anchoring device of FIG. 5A applied to the data logger device.

### DETAILED DESCRIPTION

A data logger (also datalogger or data recorder) is an electronic device that records data over time either with a built-in instrument or sensor or via external instruments and sensors. Increasingly, but not entirely, they are based on a digital processor (or computer) and called digital data loggers. Generally, they are small, battery-powered, portable, and equipped with a microprocessor, internal memory for data storage, and sensors. Some data loggers interface with a personal computer and use software to activate the data logger and view and analyze the collected data, while others have a local interface device (keypad, LCD) and can be used as a stand-alone device.

Non-limiting examples of data loggers for use with the anchoring devices of the present disclosure include (DST centi-ACT, DST centi-HRT, DST centi-HRT ACT, DST micro-ACT, DST micro-HRT, DST microRF-HRT, DST microRF-T, DST micro-T, milli-ACT, DST milli-HRT, DST milli-HRT ACT, DST Milli-T, DST nanoRF-T, or DST nano-T (STAR ODDI®)).

In some embodiments, the anchoring device is configured to receive a data logger disposed therein, wherein the data logger has a cylindrical shape. The data logger may have a side surface comprising a side surface area parallel to a central longitudinal axis of its cylindrical shape, and two end surfaces opposing each other perpendicular to the central longitudinal axis.

In some embodiments, the anchoring device has a length of about 15 mm to about 50 mm. For example, the anchoring device may a length of about 15 mm, about 20 mm, about 25 mm, about 30 mm, about 35 mm, about 40 mm, about 45 mm, or about 50 mm.

In some embodiments, the anchoring device has an external diameter of about 5 mm to about 15 mm. For example, the anchoring device may have an external diameter of about 5 mm, about 10 mm, or about 20 mm.

In some embodiments, a body of an anchoring device may be a sleeve configured to hold a data logger in place (e.g., stationary relative to the body) by wrapping around at least portion of the circumference of the data logger. A sleeve may allow the anchoring device to accommodate data loggers of a variety of shapes. For example, the body of the anchoring device may accommodate a data logger having protrusions at one or more ends of the data logger and/or along the body of the data logger.

In some embodiments, a body of a data logger anchoring device may be elongated and elastic (e.g., formed of an elastic material) and may be configured to hold data loggers with irregular features and/or curved along the length of the data logger. For example, a sleeve may be initially elastically deformed from an unstressed state such that a data logger may be inserted within the sleeve. After the data logger has been placed inside, any force being exerted on the sleeve to elastically deform the sleeve may be released, causing the sleeve to attempt to return to its original shape and dimensions. However, in some embodiments, the shape and/or dimensions of the data logger may be different from the initial unstressed shape and/or dimensions of the sleeve of the anchoring device. For example, an external diameter of the data logger may be greater than an initial unstressed diameter of a cavity of the anchoring device prior to being inserted within the anchoring device, and as such, the anchoring device may fail to return to its original dimensions and instead will shrink as much as possible and will conform to the dimensions of the data logger. Such conformation between the anchoring device and the data logger may also occur if the data logger comprises irregular features. The anchoring device may attempt to return to its original unstressed dimensions as much as possible bound by the geometry of the data logger, and as such, conform to the geometry of the data logger in the process. As the sleeve-like anchoring device is configured to conform around the shape of the data logger, a longer sleeve may offer increased friction that prevents any slippage of the data logger from the cavity of the anchoring device.

In some embodiments, a data logger anchoring device may comprise a single continuous body shaped as a sleeve. In other embodiments a, a data logger anchoring device may include a body having multiple sections each configured to wrap around the data logger.

In some such embodiments, the multiple sections may be joined together by a length of material along the central axis of the data logger. In some embodiments, the multiple sections may also be separate and/or spaced from one another when attached to a data logger. In some embodiments, the anchoring device may extend over at least a portion of a top and/or bottom of a data logger.

In some embodiments, an anchoring device may extend along greater than or equal to 25%, 50%, 75% of a total length of the data logger. An anchoring device may extend along less than 100%, 75%, 50% of the total length of the data logger in other embodiments. In some embodiments, the inventors have appreciated the benefits of a data logger that is configured to extend along at least 25% of a total length of a data logger. Such a length may provide appropriate contact area between an anchoring device and a data logger to resist removal of the data logger from the anchoring device.

In some embodiments, an anchoring device may extend around greater than or equal to 25%, 50%, 75% a circumference of the data logger. The anchoring device may also extend around less than 100%, 75%, 50%, 25% of the circumference of the data logger in other embodiments. In some embodiments, the inventors have appreciated the benefits of a data logger that is configured to extend along at least 50% of a circumference of a data logger. Such a circumference may provide appropriate contact area between an anchoring device and a data logger to resist removal of the data logger from the anchoring device.

In some embodiments, an anchoring device may be configured to cover greater than 25%, 50%, 75% a surface area (e.g., side surface area) of the data logger. The anchoring device may also cover less than 100%, 75%, 50%, 25% of the surface area of the data logger in other embodiments. In some embodiments, the inventors have appreciated the benefits of a data logger that is configured to cover less than half (e.g., 50%) of a side surface area of a data logger. Such an arrangement may allow a data logger to be more easily observed while anchored to tissue. Additionally, such an arrangement may ensure sensors of the data logger are exposed to the surrounding environment and are not blocked by the anchoring device.

Anchoring devices extending along difference portions of the length and circumference of a data logger may allow the data loggers to be adapted for a wide range of applications. In small animal species, the inventors have recognized the advantages associated with using an anchoring device with less coverage of the data logger to create minimal interference with natural biological systems of the animal and minimize adverse reactions. For example, the human small intestine may be an average of 2.5-3 cm in diameter.

As such, even a 1.0 mm thick sleeve of the anchoring device may significantly affect the movement of material within a human small intestine. For example, such an arrangement may represent a 3% to 8% increase in ratio of width of the data logger and anchoring device to the overall width of the human small intestines. Data loggers are also commonly used in mice, where any change in size may drastically affect suitability for long term implantation.

Alternatively, in some environments, the inventors have appreciated that it may be beneficial to have an anchoring device with increased coverage of the data logger to provide greater protection to the data logger against varying biological environments. Some animals, such as scavengers, comprise digestive systems with strong stomach acids, so an anchoring device with greater coverage may prevent the data logger from breaking and/or slow the rate of breakdown of the data logger to increase the amount of collected data.

In some embodiments, the body of the anchoring device is formed of an elastic material such that the body may be deformed to position the data logger in the cavity. In some embodiments, the body is made from a flexible polymer. Non-limiting examples of flexible polymers include Ethylene Vinyl Acetate, Polyethylene, Polyethylene based Polyolefin Elastomers, Polypropylene, Styrene Butadiene Copolymer, Thermoplastic Polyester Elastomer, Polypropylene based elastomers, Thermoplastic Polyurethane Elastomer, and Thermoplastic Vulcanizate. Preferably, the flexible polymer is non-toxic. In some embodiments, the material of the anchoring device may be translucent and/or translucent such that the condition of the data logger disposed within the anchoring device and the surrounding environment can be observed.

In some embodiments, the material of the anchoring device may be a thermoplastic polymer. In some such embodiments, an initial diameter of the cavity of the anchoring device in an unstressed state may be greater than an external diameter of the data logger. As such, according to such embodiments, after the anchoring device has been placed appropriately on the data logger, the anchoring device may be subject to heat which causes the thermoplastic polymer material to shrink and form an interference fit (e.g., friction fit) with the data logger. After shrinking, the diameter of the cavity of the anchoring device may equal the external diameter of the data logger.

In some embodiments, the anchoring device may form an interference fit with the data logger. In some such embodiments, the external diameter of the data logger may be larger than a diameter of the cavity of the anchoring device. As such, the anchoring device may be elastically deformed to position the data logger within the cavity of the anchoring device.

After the anchoring device is positioned around the data logger, the anchoring device may compress tightly around the data logger as the anchoring device attempts to return to its original dimensions and/or shape. After the data logger is positioned within the anchoring device, the diameter of the cavity of the anchoring device may be equal to the external diameter of the data logger. The anchoring device may exert a normal force at any point of its interaction with the data logger and increases the force of friction and form an interference fit between the anchoring device and the data logger. The inventors have appreciated that a close interference fit may additionally prevent accumulation of waste products and/or growth of bacteria in the interference between the data logger and the anchoring device.

In some embodiments, the tightness of the interference fit between the data logger and the anchoring may determine at least in part the length of the anchoring device. The contact area between an anchoring device and a data logger due to an interference/friction fit may be referred to as an interference/friction interface. For example, depending on the tightness of the friction interface, the anchoring device may cover less than or equal to 25% of the total length of the data logger. The tightness of the friction interface may be determined through an interference pressure between the anchoring device and the data logger, a friction force between the anchoring device and the data logger, and/or a transmission torque between the anchoring device and the data logger. The interference pressure, friction force, and/or transmission torque may be determined by the dimensions and/or shapes of the anchoring device and the data logger, the Young's modulus of the anchoring device and the data logger, the Poisson's ratio of the anchoring device and the data logger, the radial interference between the anchoring device and the data logger, and/or the friction coefficient of materials the data logger and/or anchoring device are made from. In some embodiments, the data logger may also be attached to the anchoring device through an adhesive, which may be used individually or in combination with an interference fit. The presence of the adhesive may additionally affect the interference pressure, friction force, and/or transmission torque between the anchoring device and the data logger.

In some embodiments, the anchoring device may be formed from a single continuous piece of material. In some cases, data loggers are often used for in vivo applications (e.g., within an animal's abdomen) and are subject to biological conditions which may encourage bacterial growth and/or accumulation of waste and/or biological products over time. As such, the inventors have appreciated that it may be advantageous to form the anchoring device out of a single continuous piece of material to avoid any crevices, holes, and any other features associated with joining multiple parts together to decrease the accumulation of waste products and/or areas for potential bacterial growth. The accumulation of waste products, biological products, and bacteria may cause harmful environments (e.g., high acidity) which may cause damage to a subject, the data logger, and/or the anchoring device. In some embodiments, a data logger anchoring device may be integrally formed by an appropriate process (e.g., molded, 3D printed, etc.)

In some embodiments, the anchoring device and the data logger disposed within may be configured to be placed within a subject and thus subjected to any movements of the subject. As such, it may be advantageous to configure the anchoring device to minimize or otherwise reduce stress concentrations to decrease any risk of failure and/or breakage. For example, joints, screws, welds, and any other applicable methods of holding multiple pieces together may cause increased stress at their locations. Combined with stresses associated with movements of the subject the data logger and the anchoring device are attached to, in some cases, failures may occur at such locations of high stress and lead to unusable and/or inaccurate data collection by the data logger. As such, a monolithic anchoring device formed from a single continuous piece of material may prove to advantageous, by avoiding the stresses associated with multiple pieces being joined together. Additionally, in some embodiments, surface features of the anchoring device may be configured with curved transitions to reduce stress concentrations at corners. For example, the anchoring portion of the anchoring device may comprise a curved transition (e.g., a slope or a curve) from the body of the anchoring device. In some embodiments, the anchoring portion of the anchoring device comprises one or more through holes formed in the anchoring portion through which sutures can be threaded. The one or more through holes may be configured to accept a suture (or multiple sutures) passing through the one or more through holes to attach the data logger to tissue of a subject. In some embodiments, the anchoring portion of the anchoring device comprises one, two, or three attachment holes. The diameter of an attachment hole, in some embodiments, is less than 1 mm, for example, about 0.9 mm, about 0.8 mm, about 0.7 mm, about 0.6 mm, about 0.5 mm, about 0.4 mm, about 0.3 mm, or about 0.1 mm. In some embodiments, an attachment hole has a diameter of about 0.1 mm to about 0.9 mm. In some embodiments, an attachment hole has a diameter of about 1 mm. In some embodiments, the anchoring portion may extend at least in part across the length of the anchoring device, such that any stress applied onto the anchoring portion from the sutures is evenly distributed.

In some embodiments, an anchoring device may comprise one or more anchoring portions. The inventors have recognized advantages associated with having one or more anchoring portions. For example, some experiments may require configurations with increased mounting stability, mounting locations, and/or any other applicable parameters that may require one or more anchoring portions. Additionally, the gastrointestinal tracts and/or any other applicable locations on an animal may be difficult to suture to due to factors including but not limited to delicate tissues and/or contorted surfaces that may require additional anchoring portions. The one or more anchoring portions may circle around a circumference of the anchoring device. The one or more anchoring devices may also extend along a length of the anchoring device parallel to the central longitudinal axis of the data logger. Combinations of the configurations described above are contemplated.

In some embodiments, the anchoring device may be composed from a single piece of continuous material that is joined at the anchoring portion. For example, the anchoring device may be integrally formed. The inventors have appreciated that in some cases it may be desirable to reduce the amount of force employed to attach an anchoring device to a data logger. Accordingly, in some embodiments the anchoring device may include a first end and a second end that form a seam. A user may move the first end away from the second end to expand the anchoring device to receive a data logger. The user may then release the first end and the second end to allow the anchoring device to return toward an unstressed state. In some embodiments, the ends of the single piece of continuous material may be joined together at an anchoring portion through adhesives, welds, thread such as a suture, and/or any other joining methods. In some embodiments, each end of the single piece of material may comprise a portion of the anchoring portion. For example, through holes of an anchoring portion may be split into two sections, with one section on each end of the single piece of material. The two sections may be attached together through methods as previously described, and suture thread may be wrapped multiple times through the through holes of the anchoring portion to further hold the two ends together.

In some embodiments, the data logger and anchoring device may be placed within a living subject (e.g., an animal's abdomen) to monitor the subject. As such, the inventors have appreciated that it may be important to maintain the health of the subject and avoid causing damage to the subject with the data logger and the anchoring device. One method of increasing safety of the anchoring device may be to configure the anchoring device with no sharp edges and/or points such that any movement of the anchoring device within a subject abdomen does not cause any damage (e.g., tears, scratches) to the surround tissue. A sharp edge and/or point may be any edge or point in which two planar surfaces meet at an appropriately narrow angle (e.g., less than 90 degrees). In some embodiments, a sharp edge and/or point may be formed by any two or more surfaces which meet and form an edge or point at an angle less than or equal to 90 degrees. In some embodiments, a sharp edge and/or point may be formed by any two or more surfaces which meet and form an edge or point at an angle less than or equal to 45 degrees.

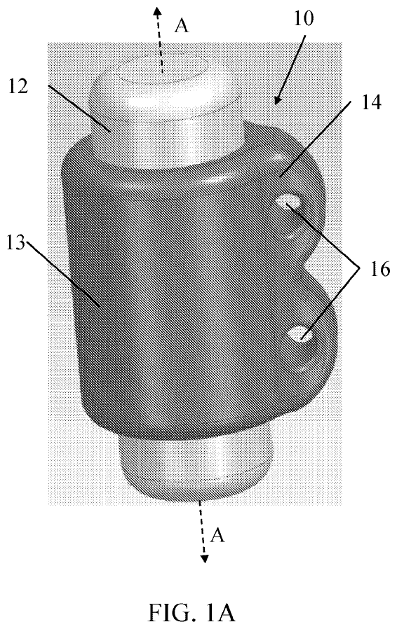

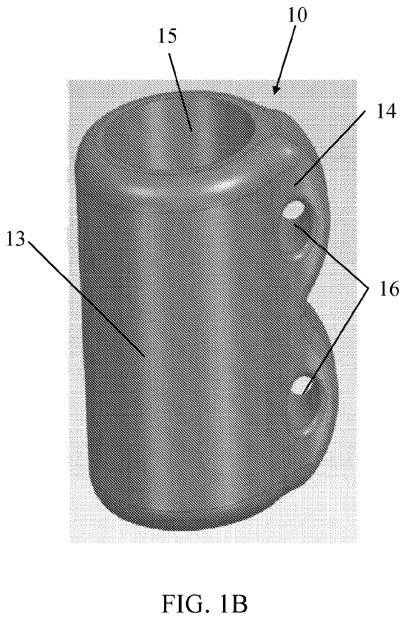

FIG. 1A shows a model of an anchoring device 10, (e.g., Prototype I) with a data logger 12 disposed within a cavity 15 of the anchoring device 10, and FIG. 1B shows a model of the same anchoring device 10 as FIG. 1A without the data logger 12 disposed within. The cavity 15 of the anchoring device 10 may be shaped like a cylinder and/or have a cylindrical shape, with a side surface area parallel to a central axis of the cylinder and/or cylindrical shape. The anchoring device 10 includes a body 13. As shown in FIG. 1A, in some embodiments the body 13 may be a sleeve that covers over half the length of the data logger 12 (e.g., measured along a longitudinal axis A of the data logger). In this embodiment, the central axis of the cavity 15 aligns with the longitudinal axis A of the data logger 12. The anchoring device 1 includes an anchoring portion 14 that stretches along an axis parallel to a central longitudinal axis A of the data logger 12 and occupies an entire length of the data logger 12. The anchoring portion 14 extends outwards from the data logger 12 in a direction orthogonal to the central longitudinal axis A of the data logger. As shown in FIG. 1A, the anchoring portion 14 includes two through holes 16 extending through a width of the anchoring portion. In some embodiments, the through holes may be able to accommodate one or more sutures passing through. In some embodiments as shown in FIG. 1A, the anchoring portion 14 conforms at least in part to the shape of the through holes 16 with curved transitions, such that minimal stress concentrations are present within the anchoring device 10. In other embodiments, an anchoring portion may include a single through hole or more than two through holes.

The anchoring device 10 may be attached to the data logger 12 through an interference/friction fit. The tightness of the friction fit may be defined by an interference pressure, friction force, and/or transmission torque at a friction interface between the anchoring device 10 and the data logger 12. The anchoring device 10 and the data logger 12 may be engaged in a friction fit through deformation of the anchoring device. In some embodiments, the anchoring device is formed of an elastic material and may be elastically deformed. For example, the body 13 of the anchoring device 10 may be elastically stretched to fit around the data logger 12. However, depending on the material of the anchoring device 10, the body 13 may also be plastically deformed to create an interference fit with the data logger 12. For example, the anchoring device may be made from a thermoplastic material. When the anchoring device is in an unstressed state the body 13 may be initially larger than the data logger 12. After the data logger has been inserted inside the anchoring device, the anchoring device may be heated to shrink around the data logger to form an interference fit. The anchoring device 10 and the data logger 12 may also be engaged in a friction fit by simply overcoming a force of friction at the friction interface. For example, the data logger and/or anchoring device may be pushed with sufficient force that the data logger is inserted into the anchoring device by overcoming frictional resistance. In some embodiments, the anchoring device and the data logger may attach at an interface through applying adhesive to the interface. However, the adhesive may also be applied to a future interface between the anchoring device and the data logger before engaging the data logger and the anchoring device through either force or deformation. Any combination of deformation, force, and adhesives may be used to attach the anchoring device to the data logger.

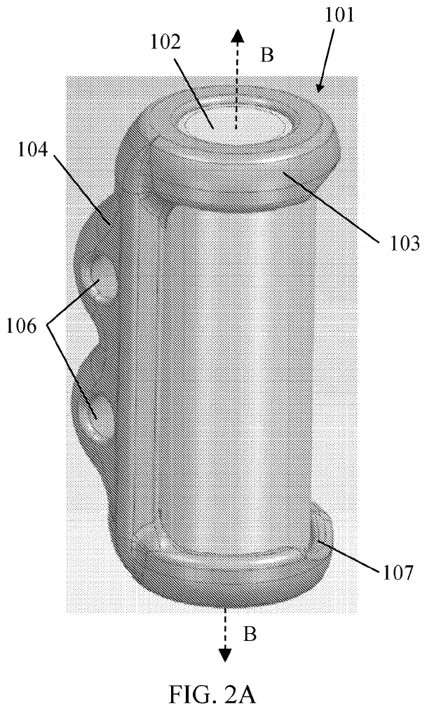

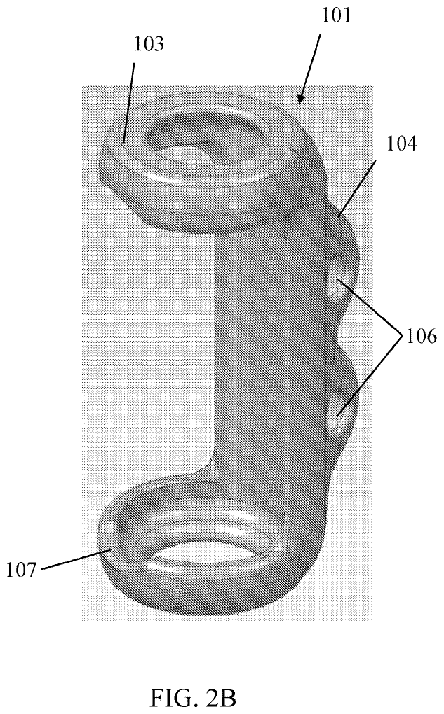

FIG. 2A shows a model of an anchoring device 101 which covers at least a part of a top and a bottom of a data logger (e.g., a Prototype II anchoring device) with a data logger 102 placed within the anchoring device, and FIG. 2B shows the same anchoring device 101 as FIG. 2A without the data logger 102 placed within. A body 103 of the anchoring device 101 may comprise a back that extends along a length of the data logger 102 (e.g., measured along a longitudinal axis B of the data logger) and comprises two opposing end portions which are situated at either end of the central longitudinal axis B of the data logger. The two end portions extend from a side surface of the data logger (e.g., the side surface parallel to the central axis of the data logger) and cover a portion of the top surface and bottom surface of the data logger (e.g., opposing top and bottom surfaces of the data logger which are orthogonal to the axis B). A portion, which may be a majority in some embodiments, of the side surface of the data logger 102 may remain uncovered by the anchoring device 101 as the data logger is held in place by the two end portions of the anchoring device. The two end portions additionally comprise divots 107 (though only the divot 107 on the bottom end portion of the anchoring device 101 is visible in FIG. 2A), which may be used to facilitate placement of the data logger 102 into the anchoring device. In this embodiment, the anchoring portion 104 comprises two through holes 106 which extends partially across the length of the anchoring device 1 parallel to the longitudinal axis B of the data logger 102. In this embodiment, the body 103 extends around less than 50% of a circumference of the data logger 102 placed within the anchoring device 101.

The data logger 102 may be inserted into the anchoring device 101 through deformation of the body 103 of the anchoring device to form a friction fit at an interface where the anchoring device and the data logger come into contact. In some embodiments, the data logger 102 may be placed in the anchoring device 101 through elastic deformation. At least one of the back, the top end portion, or the bottom end portion of the body 103 may elastic deform such that there is sufficient space for the data logger 102 to be disposed within the anchoring device 101. The divots 107, as previously mentioned, may increase an ease of entry of the data logger 102 by increasing a clearance between the data logger and the end portions of the body 103. In some embodiments, the data logger 102 may also be placed in the anchoring device 101 through plastic deformation. For example, the anchoring device may be made of shrinkable material (e.g., thermoplastic materials). The data logger 102 may be aligned properly within an initially larger anchoring device 101, which will shrink to fir the data logger upon application of heat.

In some embodiments, data loggers may comprise domed, curved, beveled, flat, or any other applicable top and/or bottom end surfaces. As such, the end portions of the body of an anchoring device may be adapted to fit data loggers with top and/or bottom surfaces of different shapes. In FIG. 2B, surfaces where the end portions of the body 103 have dome-shaped cavities, which may help hold a domed, beveled, and/or curved data logger in place.

For example, a dome-shaped cavity may be configured to receive a correspondingly dome-shaped end surface of a data logger. In other embodiments, an end portion of a data logger anchoring device may including a flat cavity configured to receive a corresponding flat end surface of a data logger. In these embodiments, “dome-shaped” or “flat” cavities may refer to a shape profile of the surface at the deepest portion of the cavity. In some embodiments, a tightness of a friction fit at an interface between end portions of an anchoring device and top and/or bottom surfaces of a data logger may have sufficient strength for an anchoring device to hold a data logger with a different end surface shape to the end portion cavities of the anchoring device. For example, an anchoring device with domed end cavities may hold a data logger with beveled end surfaces in place.

In this embodiment, the end portions comprise a hole, which may allow parts of a data logger disposed within to protrude. However, it may also be possible that any end portions of a body of an anchoring body do not comprise an opening.

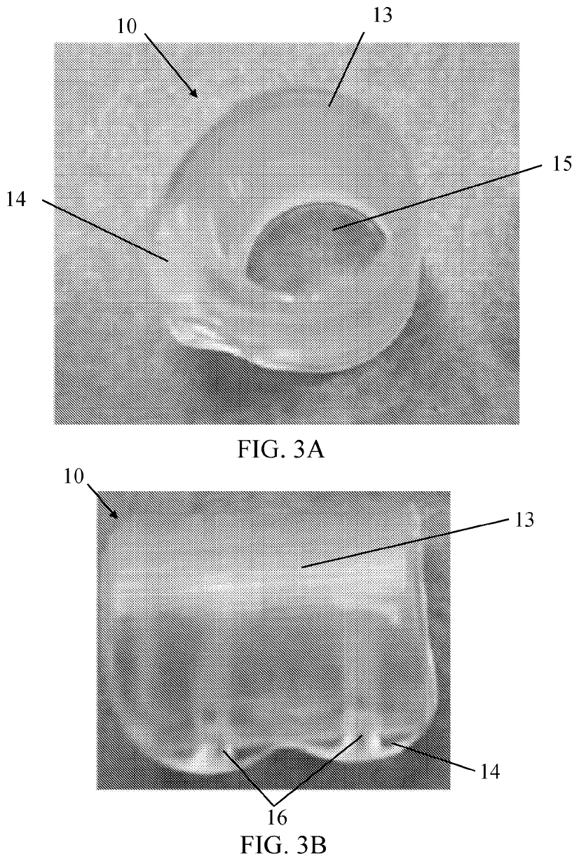

FIG. 3A shows a Prototype I anchoring device 10 made from a translucent material from a top perspective. The body 13 of the anchoring device 10 is shaped as a symmetrical circular tube, and the cavity 15 is shaped as a cylinder. The anchoring portion 14 protrudes outwards from the body 13 of the anchoring device 10.

FIG. 3B shows the anchoring device 10 of FIG. 3A from a side perspective. From this angle, through holes 16 which extend through a width of the anchoring portion 14 of the anchoring device 10 can be observed.

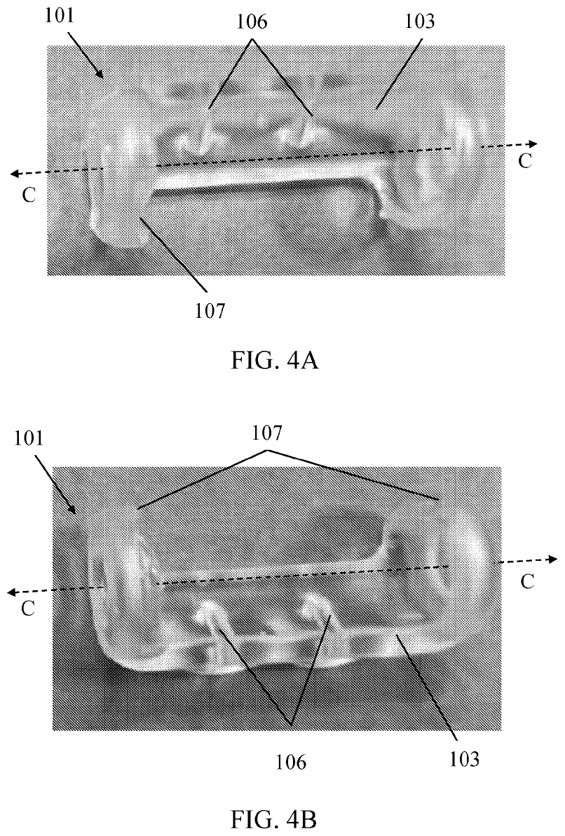

FIG. 4A and FIG. 4B shows a Prototype II anchoring device 101 made from a translucent material in two different side perspectives. The body 103 of the anchoring device 101 extends along a longitudinal axis C of the device. In this embodiment, a back of the body 103 extends around less than 50% of a circumference of any data logger that may be placed within the anchoring device 101. The body 103 comprises two end portions, which are at opposing ends of the longitudinal axis C, wherein each end portion comprises an opening that may allow top and/or bottom surfaces of a data logger disposed within to protrude through. The end portions have divots 107 which may facilitate placement of a data logger within the anchoring device 101. The body 103 comprises an anchoring portion which extends outwards from the body, but due to the angle of the photographs, the anchoring portion cannot be observed. However, through holes 106 which extend through a width of the anchoring portion can be seen.

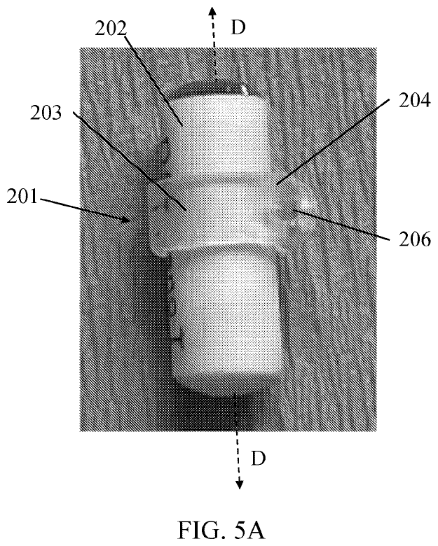

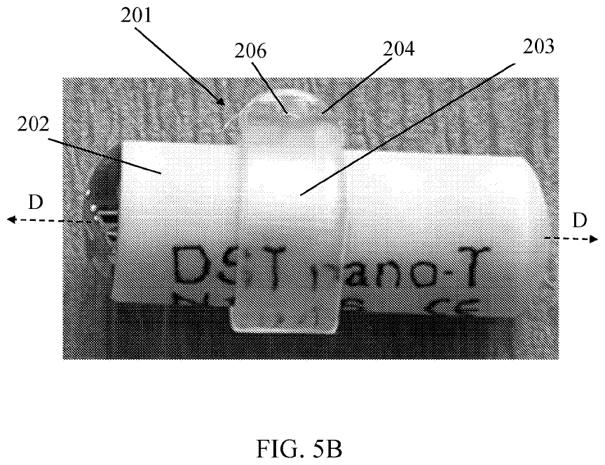

FIG. 5A and 5B show a Prototype III anchoring device 201 which covers around 25% of the length of the data logger 202 disposed within. In some embodiments, the inventors have appreciated that an anchoring device extending along 25% and 50% of the length of the data logger may be advantageous to reduce biological tissue reactions to the anchoring device. The anchoring portion 204 extends outwards from the data logger 202 and/or body 203 of the anchoring device 201 and comprises a single through hole 206. The inventors have appreciated that in some cases, a single through hole may be advantageous in reducing the number of sutures to secure the anchoring device to tissue. The anchoring device 201 may be disposed at or around a midpoint of a length of the data logger 202 along a longitudinal axis D of the data logger and/or a geometric centroid. In some embodiments, a data position of an anchoring device on a data logger may be determined at least in part by a geometric centroid and/or a center of mass of the data logger. For example, a center of mass of the data logger may not align with the geometric centroid of the data logger, which may change an engagement location of the anchoring device on the data logger. As shown in FIGS. 5A and 5B, the anchoring device 201 is located slightly away from the midpoint along the longitudinal axis D. For example, the inventors have appreciated that placing the anchoring device approximately 1/3 along the length of the data logger (e.g., ⅓ of the length in a direction away from the proximal, clear end of the data logger) may be beneficial. In this embodiment, the body 203 is a continuous piece of material, which is elastically and/or elastically deformed to receive the data logger 202 in a friction fit. However, in some embodiments, the body may be closed around the data logger 202 through the aid of one or more sutures which may pass and/or loop through the through hole 206. For example, the through hole 206 may be split into two sections at a seam and may be joined together by the one or more sutures. In some embodiments, the two sections of anchoring portion 204 may be joined by adhesive. In some embodiments, each of the sections of the anchoring portion may be disposed on opposing ends of the body 203, and the opposing ends may be moved away from each other to expand the cavity of the anchoring device to more easily receive the data logger 202. In some embodiments, an interface between the anchoring device 201 and the data logger 202 may also be solely based on an interference fit and/or a combination of sutures, friction, and/or adhesive.

In some embodiments, multiple Prototype III anchoring devices may be used for a single data logger. In some embodiments, a combination of Prototype I, Prototype II, and/or Prototype III anchoring devices may be used for a single data logger.

### EXAMPLES

### Example 1

Three prototypes of the anchoring devices (FIGS. 1A-5B) were produced and tested in mice. When comparing necropsy outcomes in mice with free-floating dataloggers and anchored dataloggers, it was generally noticed that the free-floating loggers had the potential to migrate to the caudal abdomen or inguinal canal, possible irritating sensitive tissues such as the urinary bladder. In one mouse, a free-floating (e.g., non-anchored) datalogger appeared to have been associated with an intestinal obstruction. Anchoring the dataloggers with the anchor in FIGS. 5A-5B prevented these complications. No data was collected from the datalogger itself during these trials.

FIGS. 5A-5B depict the inventor's preferred prototype for future studies in mice. While testing the prototypes in mice, it was determined that the design with the least amount of material (FIGS. 5A-5B) was best as it resulted in the least amount of biological tissue response. The prototype in FIGS. 5A-5B also saves time due to only having to suture one and requires a slightly smaller surgical incision compared to the other prototypes. Below are the details from trials conducted with various embodiments of data logger anchoring devices according to exemplary embodiments described herein.

### Free-floating Datalogger Implanted in a Horizontal (Medial to Lateral) Orientation

Ten C57B1/6J female mice, 15 weeks old, body weight 20-26 grams were implanted with DST nano-T in the horizontal (transverse) orientation along the midline of the lower abdomen. Implantation was followed by 9 days of post-op observation, euthanasia, and then gross visual examination. Mice were anesthetized with isoflurane (˜15 minutes) and long-acting buprenorphine analgesia was administered for analgesia. Incisions were made through the skin and abdominal wall along the flank (fur shaved and skin aseptically prepared prior to incisions). DST nano-T was placed within the abdominal cavity in the transverse orientation. The abdominal wall was closed with absorbable suture and the skin was closed with a wound clip. Subcutaneous saline and carprofen were administered post operatively.

Results: One mortality was observed on post-op Day 2. Gross necropsy indicated that the mortality was due to the DST nano-T having migrated and caused an intestinal obstruction. Nine animals recovered normally following implant. On gross necropsy, in all 9 animals the dataloggers had slipped caudal and ventral within the peritoneal cavity but and remained in the horizontal orientation. Increased vascularization within the mesenteric fat was noted where the dataloggers were in contact with fat pads.

Conclusion: Horizontal orientation of DST nano-T implant would not be repeated. Instead, DST nano-T would be placed in the vertical (cranial-caudal) plane.

### Free-floating Datalogger Implanted in a Vertical (Cranial to Caudal) Orientation

Five female and 5 male, C57B1/6J mice, 6 weeks old, body weight: ˜ 18 g female, ˜22 g male were implanted with DST nano-T in the vertical (cranial-caudal) orientation along the flank of the lower abdomen. Implantation was followed by 4-6 weeks of post-op observation, euthanasia, and then gross visual examination.

Methods: Mice were anesthetized with isoflurane (˜15 minutes) and long-acting buprenorphine analgesia was administered for analgesia. Incisions were made through the skin and abdominal wall along the flank (fur shaved and skin aseptically prepared prior to incisions). DST nano-T was placed within the abdominal cavity in the cranial-caudal orientation along the flank. The abdominal wall was closed with absorbable suture and the skin was closed with a wound clip. Subcutaneous saline and carprofen were administered post operatively.

Results: On post-op Day 3, 1 female was euthanized due to incisional chewing, and the other 9 mice recovered normally following implant. Upon gross exam, 1 out 4 female mice and 3 out of 5 male mice were observed to have bladder thickening. In 2 out of 5 male mice, the datalogger had migrated caudally into the inguinal canal.

Conclusion: The bladder thickening was hypothesized to be due to contact of the cranial pole of the bladder with the caudal end of the DST nano-T.

### Trial of Datalogger Anchoring Device Prototypes: Larger Ring Design (FIGS. 3A-3B) and Clamp Design (FIGS. 2A-2B)

C57B1/6J mice, n#, age, sex, and body weight unknown with implanted with DST nano-T with a datalogger anchor in the vertical (cranial-caudal) orientation along the ventral midline of the lower abdomen. Implantation was followed by 2 weeks of post-op observation, euthanasia, and then gross visual examination. Two different anchoring device embodiments were tests: 1) medical grade resin flexible medium sized ring (FIGS. 3A-3B) and medical grade resin clamp (FIGS. 2A-2B).

Methods: Mice were anesthetized with isoflurane (˜20 minutes) and long-acting buprenorphine was administered for analgesia. Ventral midline incisions were made through the skin and abdominal wall (fur shaved and skin aseptically prepared prior to incisions). DST nano-T was placed within a suture tab. DST nano-T was placed within the abdominal cavity parallel to the ventral midline incision. Non-absorbable nylon was used to anchor the suture tab to the ventral abdominal wall. The abdominal wall was closed with absorbable suture and the skin was closed with a wound clip. Subcutaneous saline and carprofen were administered post operatively.

Results: All mice recovered normally following implant. Upon gross exam, vascularization was observed-vascularized fat encapsulated the DST nano-T.

Conclusion: Tethering added more material into the body and one animal showed an adverse impact to the intestine tissue, but the suture did inhibit contact with the bladder (advantage). The exemplary anchoring device of FIGS. 5A-5B was developed to refine the suture tab design to reduce the amount of resin material required for the tether with the goal of reducing the biological tissue responses.

### Trial of Datalogger Anchoring Device Prototype: Clamp Design (FIGS. 5A-5B)

Three C57B1/6J mice, 10 weeks old, body weight and sex unknown were implanted with implanted with a DST nano-T datalogger and resin clamp anchor in the vertical (cranial-caudal) orientation along the ventral midline of the lower abdomen. Implantation was followed by 11 days post-op observation, euthanasia, and then gross visual examination.

Methods: Mice were anesthetized with isoflurane (˜20 minutes) and long-acting buprenorphine analgesia was administered for analgesia. Ventral midline incisions were made through the skin and abdominal wall (fur shaved and skin aseptically prepared prior to incisions). DST nano-T was placed within the anchor such that the anchor was located at approximately ⅓ of the length of the data logger away from the proximal, clear end of the data logger. DST nano-T was placed within the abdominal cavity parallel to the ventral midline incision. Non-absorbable nylon was used to secure the anchor to the ventral abdominal wall. The abdominal wall was closed with absorbable suture and the skin was closed with a wound clip. Subcutaneous saline and carprofen were administered post operatively.

Results: All mice recovered normally following implant. Upon gross exam, all of the data loggers were secure within the suture tab. There was minimal tissue reaction to the data logger and suture tab.

Conclusion: Tethering with a smaller size anchor (FIGS. 5A-5B) may reduce biological tissue reactions caused by free-floating data loggers and larger anchors.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean +10% of the recited numerical value.

Where a range of values is provided, each value between and including the upper and lower ends of the range are specifically contemplated and described herein.

What is claimed is:

1. A data logger anchoring device comprising:
a body including a cavity configured to receive a data logger disposed therein;
an anchoring portion attached to the body; and
one or more through holes formed in the anchoring portion, wherein the one or more through holes are configured to accept a suture passing therethrough to attach the data logger anchoring device to tissue of a subject.

2. The data logger anchoring device of claim 1, wherein the cavity is a cylindrical cavity extending through the body, and wherein the cavity is configured to form a friction fit with the data logger.

3. The data logger anchoring device of claim 1, further comprising the data logger disposed in the cavity.

4. The data logger anchoring device of claim 1, wherein the anchoring portion extends outwards from the body, and wherein the one or more through holes are a single through hole.

5. The data logger anchoring device of claim 1, wherein the body holds the data logger stationary relative to the body.

6. The data logger anchoring device of claim 1, wherein the cavity in an unstressed state has a diameter that is less than an external diameter of the data logger.

7. The data logger anchoring device of claim 6, wherein the data logger anchoring device is formed of an elastic material which is elastically deformed to receive the data logger.

8. The data logger anchoring device of claim 1, wherein the cavity of the data logger anchoring device has a diameter greater than an external diameter of the data logger.

9. The data logger anchoring device of claim 8, wherein the data logger anchoring device is made from a thermoplastic material which is configured to be plastically deformed after receiving the data logger to reduce the diameter of the cavity.

10. The data logger anchoring device of claim 1, wherein the data logger anchoring device is formed of a single piece of continuous material.

11. The data logger anchoring device of claim 1, wherein the body of the data logger anchoring device is configured to extend along between 25% and 50% of a total length of the data logger.

12. The data logger anchoring device of claim 1, further comprising end portions attached to the body, wherein the end portions cover at least a portion of a top surface and a bottom surface of the data logger.

13. The data logger anchoring device of claim 12, wherein the data logger is a cylinder having a side surface with a side surface area, wherein the body of the data logger anchoring device is configured to cover less than or equal to half of the side surface area.

14. The data logger anchoring device of claim 12, wherein each of the end portions of the data logger anchoring device comprise a dome-shaped cavity configured to receive a dome-shaped end surface of the data logger.

15. The data logger anchoring device of claim 12, wherein each of the end portions of the data logger anchoring device comprise a flat cavity configured to receive a flat end surface of the data logger.

16. The data logger anchoring device of claim 1, wherein the data logger anchoring device comprises no sharp edges and/or points.

17. A data logger anchoring device comprising:
a body including a first end and a second end configured to join together when disposed around a data logger;
an anchoring portion attached to the body; and
one or more through holes formed in the anchoring portion, wherein the one or more through holes are configured to accept a suture passing therethrough to attach the data logger anchoring device to tissue of a subject.

18. The data logger anchoring device of claim 17, wherein the data logger anchoring device is formed of a single piece of continuous material.

19. The data logger anchoring device of claim 17, wherein the anchoring portion is disposed on at least one of the first end and the second end.

20. The data logger anchoring device of claim 19, wherein the anchoring portion has a first section disposed on the first end, wherein the anchoring portion has a second section disposed on the second end, and wherein the first section and second section are configured to join together to form the anchoring portion.
